(19) United States
(12) Plant Patent Application Publication
(10) Pub. No.: US 2019/0281742 P1
(43) Pub. Date: Sep. 12, 2019

(54) Citrus sinensis plant named 'ROSALINA'

(50) ;

(71) Applicant: Glenam Knott, Fort Beayfort (ZA)

(72) Inventor: Glenam Knott, Fort Beayfort (ZA)

(21) Appl. No.: 15/932,522

(22) Filed: Mar. 9, 2018

(51) Int. Cl. A01H 6/78

(52) U.S. Cl. PLT/202

(57) ABSTRACT

A new and distinct navel orange distinguished by production of fruit that combines early season maturity and a dark orange to red pigmented rind, (UPOV Group 20), with a pink albedo. It further distinguishes itself with a deep orange red flesh, that matures 3-4 weeks before Cara-Cara navel, the current bench-mark red navel. Both flesh and juice are consistently darker than that of Cara-Cara. The new cultivar is a Citrus sinensis, useful for commercial fruit production.

Latin name of the genus and species: Citrus sinensis.

Title: Orange Tree Named ‘Rosalina’.

### BACKGROUND OF THE INVENTION

The new variety ‘Rosalina’ originated as a spontaneous branch mutation discovered by Glenam Knott in an orchard of Citrus sinensis ‘Lina’. The discovery was made during 2009 in Eastern Cape, South Africa. On 3rd Oct. 2009, plant material was selected from the mutation branch for clean up at the Citrus Research International in Nelspruit. The is an extended and confidential process. In addition to the cleanstock production, in February 2010 the inventor began assessing the new variety confidentially at his own orchard in Eastern Cape, South Africa. Buds from the mutation branch were top grafted onto ‘Swingle’ rootstock with Cara-Cara Navel interstock in February 2010. The interstock was employed to improve compatibility due to ‘Lina’ navel's slight incompatibility with ‘Swingle’ rootstock. Fruit production on these trees commenced on 2013. Additional propagation of ‘Rosalina’ direct onto Swingle rootstock were made at the same site to replace failed top grafts in 2013.

Various instabilities, including foliar variegation were observed. After 6 generations a final selection was made of a stable clone.

### SUMMARY OF THE INVENTION

The cultivar ‘Rosalina’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Rosalina’. These characteristics in combination distinguish ‘Rosalina’ as a new and distinct Citrus sinensis cultivar:
1. Early season maturity.
2. Dark orange pigmented rind, occasionally with red blush.
3. Red pigmented flesh.
4. Red pigmented albedo.
5. Round, small fruit shape, less oblong.
6. Higher percentage of the fruit have a less protruding navel end.

### COMPARISON TO PARENT VARIETY

Plants of the new cultivar ‘Rosalina’ are similar to plants of the seed parent, Citrus sinensis ‘Lina’ in most horticultural characteristics, however, plants of the new cultivar ‘Rosalina’ differs in the following ways;
1. New cultivar's growth shape is more spherical and more compact than parent variety.
2. Flesh of new cultivar's fruit is red pigmented; parent's flesh is yellow-orange.
3. New cultivar's rind color is a darker orange than that of parent.
4. New variety has red flecked albedo, whereas parent has white albedo.
5. New variety has a thinner, but stronger rind than parent.

### COMMERCIAL COMPARISON

The new variety is best compared to the commercial variety, ‘Cara-Cara’. ‘Rosalina’ is similar to ‘Cara-Cara’ in many horticultural characteristics; however, ‘Rosalina’ differs in the following;
1. Fruit flesh of ‘Rosalina’ consistently has a darker red pigment than the red-pink comparator.
2. The rind color of ‘Rosalina’ is a darker orange than that of Cara-Cara.
3. A high percentage of fruit of ‘Rosalina’ have red blushed rinds, a characteristic completely absent in Cara-Cara.
4. The new variety has red flecked albedo, whereas comparator has white albedo.

### BRIEF DESCRIPTION OF THE PHOTOGRAPHS

The accompanying photographs illustrates in full color examples of typical fruit harvested from ‘Rosalina’ and comparators.

FIG. 1 illustrates a comparison of the calyices ‘Rosalina’ and ‘Cara Cara’ on slightly immature fruit.

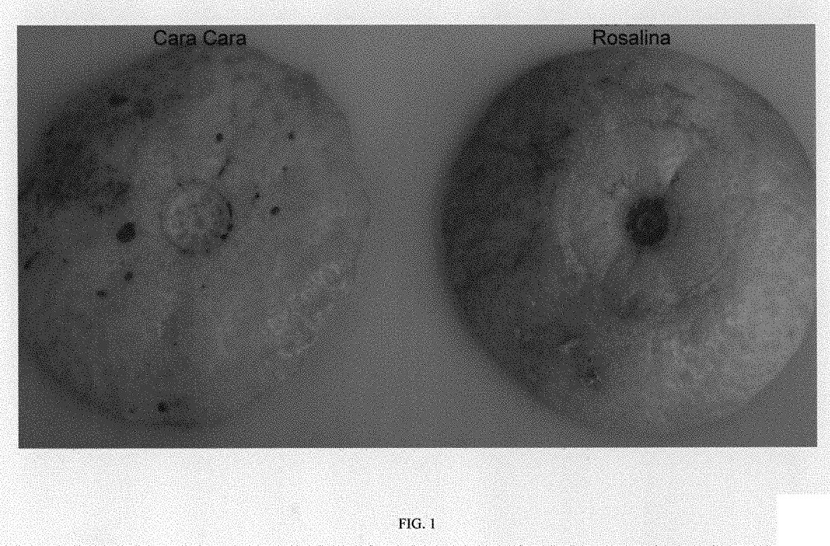

FIG. 2 illustrates a comparison of the calyices ‘Rosalina’ and ‘Lina’ on slightly immature fruit.

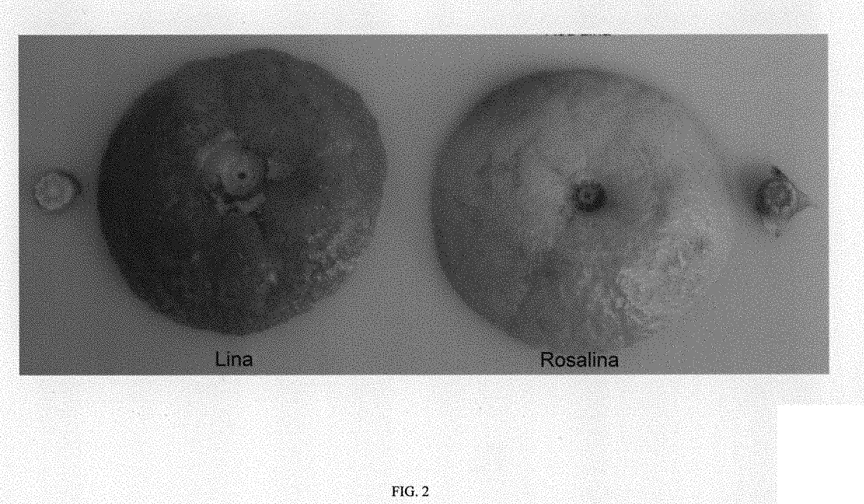

FIG. 3 illustrates a lengthwise comparison of ‘Cara Cara’, ‘Lina’ and ‘Rosalina’.

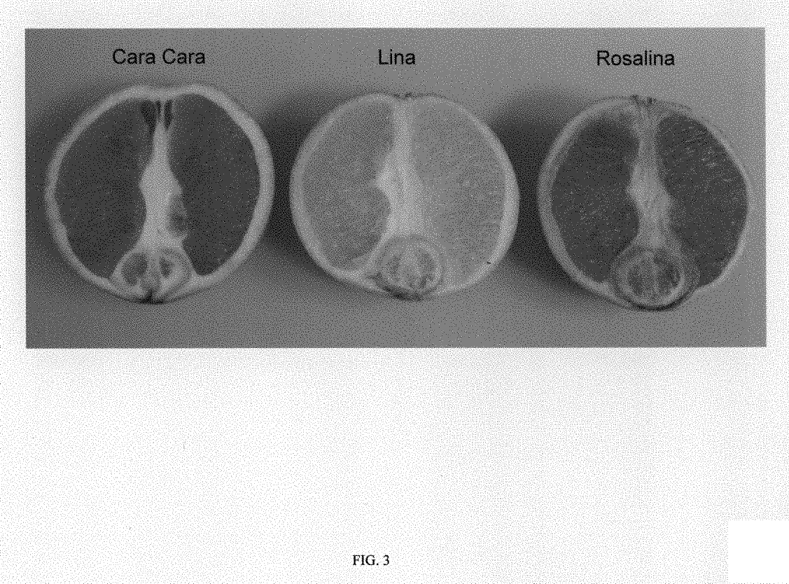

FIG. 4 illustrates albedo of ‘Cara Cara’, ‘Lina’ and “Rosalina’.

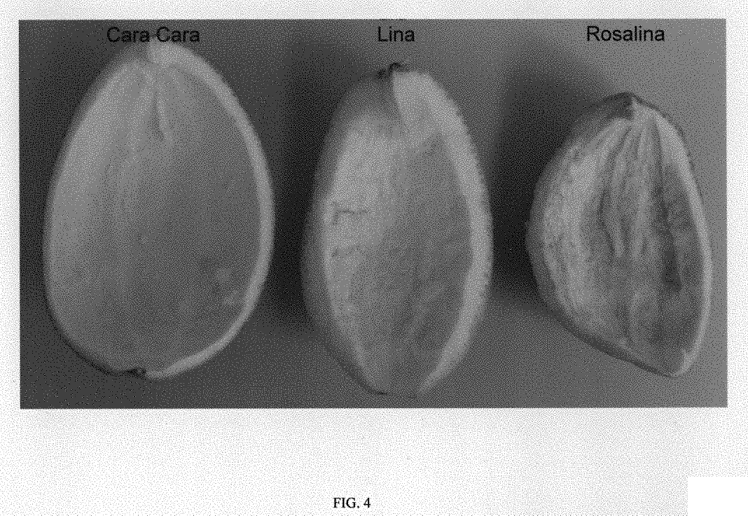

FIG. 5 illustrates a hemispherical cross -section of ‘Cara Cara’, ‘Lina’ and ‘Rosalina’.

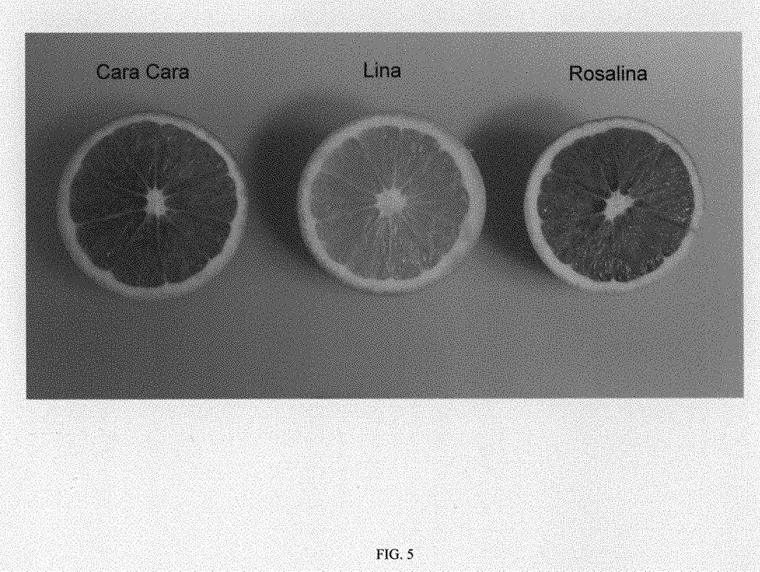

FIG. 6 illustrates a seven-year-old top grafted tree of ‘Rosalina’ on navel interstock onto Swingle rootstock.

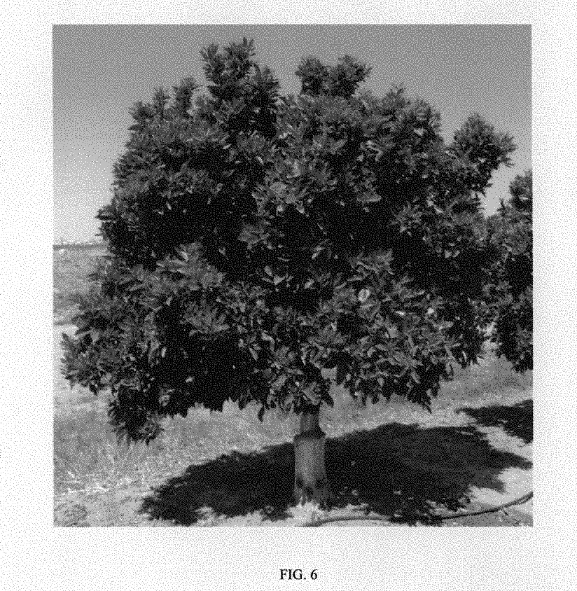

FIG. 7 illustrates ‘Rosalina’ albedo and flesh color in comparison with the comparator variety Cara-Cara Navel.

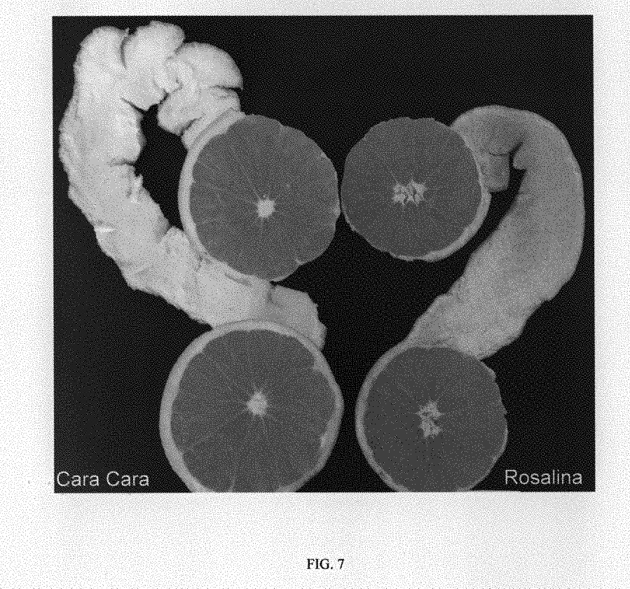

FIG. 8 illustrates ‘Rosalina’ rind color in comparison with comparator variety ‘Cara-Cara’.

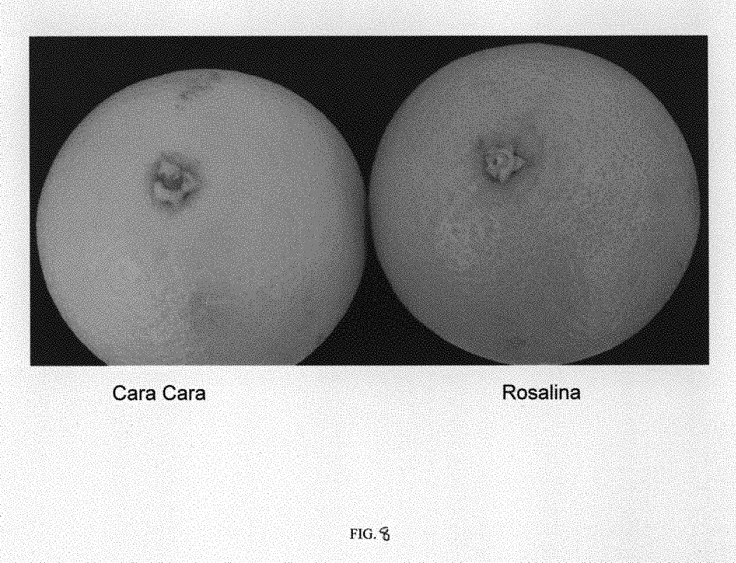

FIG. 9 illustrates flowers and foliage of ‘Rosalina’.

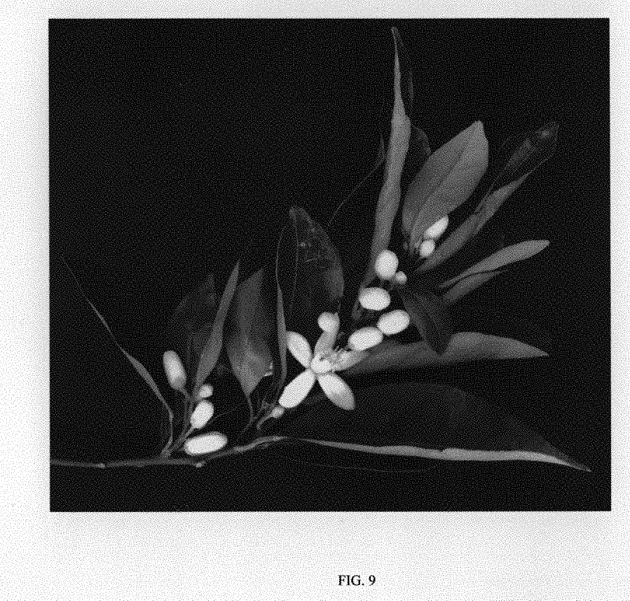

FIG. 10 illustrates a fruiting tree of ‘Rosalina’ at approximately 7 years.

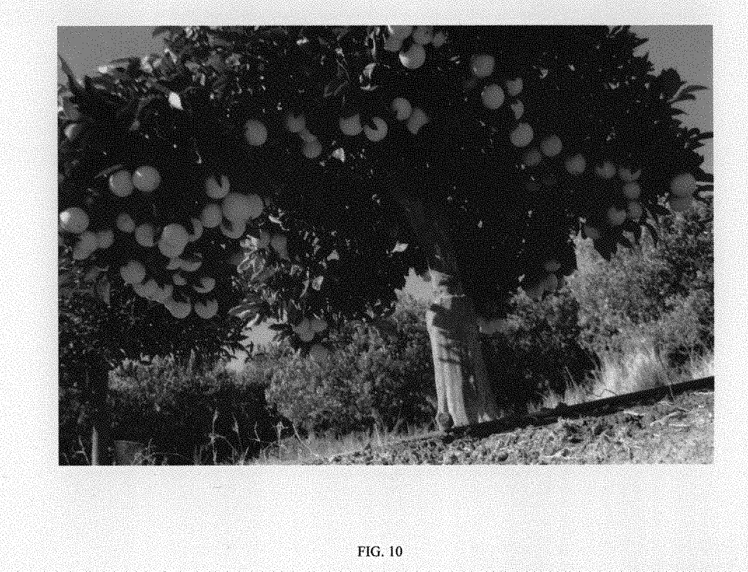

The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘Rosalina’ plants grown outdoors under commercial trial conditions in the Cape of South Africa. Measurements and numerical values represent an average from the most typical specimens.
Botanical classification: Citrus sinensis ‘Roslina’.

### PROPAGATION ‘Rosalina’ is typically grafted onto ‘Swingle’ rootstock.

### PLANT

Age of the plant described: Approximately 7 years.
Plant habit: Upright.
Height: Approximately 2.5 m.
Growth rate: Approximately 3 years to reach 185 cm.
Plant spread: Approximately 2 m.
Length of primary lateral branches: Approximately 52-72 cm.
Quantity of lateral branches: 2-4.
Angle of lateral branches from main trunk: 150-170° degrees.
Branching arrangement: Typically 2-3 more vertical main limbs with 1-2 smaller and more horizontal shoots. The vertical shoots rebranch after 52-72 cm.
Branch diameter: Average 3.8-5.0 cm.
Trunk texture: Minimally rough, slightly striated.
Trunk color: Near RHS Grey-Brown N199A.
Trunk diameter: Average 8.0 cm on a 3 year old plant.
Spines:
   Density.—Absent or sparse.

### FOLIAGE

Leaf:
   Average length.—Range: 6.5-8.25 cm. Average of 7.6 cm.
   Average width.—Range: 2.8-3.9 cm. Average of 3.2 cm.
   Shape of blade.—Elliptic.
   Apex.—Acute.
   Base.—Broad attenuate.
   Attachment.—Petiolate.
   Margin.—Entire to crenate. Absent or week undulation.
   Texture of top surface.—Smooth.
   Texture of under side.—Smooth with raised center rib.
   Aspect.—Foliage typically upwardly folded. No twisting or blistering with normal cultural conditions.
   Color.—Mature foliage upper side: Near RHS Green 137A. Mature foliage under side: Near RHS Green 138A.
   Petiole.—Length: Average length 0.75-1.6 cm. Width: Average 0.3 cm. Pubescence: None. Color: Upper and lower surfaces near Green 143B. Petiole Wing: Approximately 7 mm length. Approximately 2 to 3 mm wide. Color and texture identical to leaf.

### FLOWER

Inflorescence and flower type and habit: Single rotate flowers emerging from leaf axils.
Flowering period: Buds form in the Eastern Cape of South Africa in Mid to Late October.
Flower longevity on plant: Approximately 2 weeks.
Persistent or self-cleaning: Self-cleaning.
Bud:
   Shape.—Ovate.
   Length.—Average cm.
   Diameter.—Average 5 mm.
   Color.—Near RHS White 155A.
Flower size:
   Diameter.—Average 2.5 cm.
   Depth.—Average 1.5 cm.
Corolla/petals:
   Arrangement.—Rotate, fused at base. Not overlapping.
   Petal quantity.—5.
   Length.—Average 1.2 cm.
   Width.—Average 4 to 5 mm.
   Texture.—Smooth without pubescence.
   Apex.—Acute.
   Base.—Fused.
   Margin.—Smooth.
Color: Petals are white upon abscission.
Calyx/sepals: Filaments separate easily.
   Quantity per flower.—5.
   Shape.—Straight.
   Length.—Average 6-7 mm.
   Width.—Average 1 mm.
   Texture.—Smooth.
   Color.—Upper Surface: Near RHS Green 137A. Lower Surface: Near RHS Green 137A.
Pedicel:
   Length.—Average 4-5 mm.
   Diameter.—Average 1 mm.
   Color.—Near RHS Green 137A.
   Texture.—Smooth without pubescence.
Fragrance: Sweet, typical Citrus flower scent.

### REPRODUCTIVE ORGANS

Stamens:
   Number.—Most commonly 12.
   Filament length.—Approximately 2.2 cm.
Anthers:
   Shape.—Globular.
   Length.—Approximately 0.2 cm.
   Color.—Near RHS Yellow 3A.
   Pollen.—Color: Near RHS Yellow 9C. Quantity: Moderate.
Pistil:
   Number.—1.
   Length.—Approximately 0.8 cm.
   Style.—Length: 0.6 cm. Color: Near RHS Green-Yellow 1D.
   Stigma.—Shape: linear. Color: Near RHS Yellow 8D. Ovary Color: RHS Green 143C.

### FRUIT

Color exocarp: Near RHS Orange 26A.
Color mesocarp: Near RHS Yellow-Orange 18D.
Flesh color: Near RHS Orange-Red 34b, 34C.
Brix at consumption: 10.4 to 11.6.
Brix at harvest: 12.5. Acid 0.88 Ratio of 10.50.
Diameter: Average diameter at peak: 75 to 85 mm.
Segment number per fruit: Medium, 10 to 11.
General shape: Round.
Fruit neck: Not present.
Stalk end depression: Present, deep.
Stalk end radial grooves: Intermediate quantity of long grooves.
Fruit collar: Absent.
Distal end depression: Absent.
Areola: Absent.
Diameter stylar scar: Large.
Persistance of style: None.
Naval opening: Always present, medium in size. Absent or weak bulging.
Skin/rind texture: Medium to rough. Pitting and pebbling present. Sparse pitting. Peddling moderate, occurring on oil glands only.
Rind: Medium thickness, medium strength.
Oil glands: Small to medium, very little difference in size. Moderate conspicuousness.
Rind oil: Moderate quantity of oil. Inner surface oil glands conspicuous.
Fruit core: Densely filled.
Rudimentary segments: Variable, from weak to intermediate.
Segment wall strength: Moderate.
Fruit juiciness: Moderate.
Seeds: Absent or very few.

### OTHER CHARACTERISTICS

Disease/pest resistance: No susceptibility nor resistance observed.
Temperature tolerance: Tolerates low temperatures to approximately −5° C. without negative effects, tolerates high temperatures to approximately 37° C. without negative effects.

What is claimed is:

1. A new and distinct Orange tree named ‘Rosalina’ as herein illustrated and described.
